(19) United States
(12) Patent Application Publication
(10) Pub. No.: US 2006/0095018 A1
(43) Pub. Date: May 4, 2006

(54) Catheter with curved distal end and method of making the same

(76) Inventor: Matt D. Pursley, Alpharetta, GA (US)

Correspondence Address: THOMPSON & THOMPSON, P.A., P.O BOX 166, SCANDIA, KS, 66966, US

(21) Appl. No.: 11/261,544

(22) Filed: Oct. 27, 2005

Related U.S. Application Data
(60) Provisional application No. 60/623,714, filed on Oct. 28, 2004.

(51) Int. Cl. A61M 25/00

(52) U.S. Cl. 604/526; 138/132

(57) ABSTRACT

A catheter having a curved distal end is disclosed in which a resilient fiber embedded in a polymer material of the sidewall imparts a bend in the catheter. The resilient fiber has a helical coil shape with a series of helical coils disposed about a center line. During manufacturing, the resilient fiber is bent into a curved condition in which the center line is curved, and then the resilient fiber is heated while in its curved condition to create a memory set in the helical coil shape. The resilient fiber is then placed over a mandrel along with a fibrous reinforcement material, and a polymer material is applied over the mandrel to form a catheter with the resilient fiber embedded in the sidewall. Upon removing the mandrel from the lumen of the catheter, the catheter will bend into a curved shape corresponding to the memory set in the resilient fiber.

### RELATED APPLICATIONS

This application claims priority of U.S. Provisional Application No. 60/623,714 filed on Oct. 28, 2004, the content of which is incorporated herein by reference.

### BACKGROUND OF THE INVENTION

1. Field of the Invention

The present invention relates generally to catheters and, in particular, to catheters that can be curved or bent at their distal ends or other selected locations, and methods for making such catheters.

2. Description of the Related Art

Catheters are generally either straight or curved. A curved catheter is generally curved during manufacturing to have a “preset” curve with a radius and location that enhances the physician's ability to introduce the catheter to the desired location. Usually, this curve is “set” in the catheter by first bending a straight catheter at ambient conditions to the desired shape, then applying heat to the polymer contained in the catheter wall while in this curved state, and then allowing the catheter to cool while still in this curved state. This process imparts a memory to the polymer in the curved state because at an elevated temperature the polymer loses its memory of being straight and gains a new memory of being in a curved position as the polymer cools while the curved shape is maintained. In some cases, the catheter can be curved without heat by cold working the catheter into a curved shape.

In use, the catheter with a preset curved shape can be straightened by a physician to enter a patient's body, usually over a guide wire. Once in the body, the catheter is advanced over the guide wire until it reaches a soft portion of the wire near the diseased section of artery, and the catheter's residual stress (from the memory set portion of the catheter curve) begins to override the straightening force of the wire and begins to take its original curved shape.

A shortcoming of this prior art method of curving a catheter is that the force available to recurve the catheter as it reaches its deployment location within a patient's body is very small, particularly with an extremely soft polymer. In cardiovascular catheters, the curved section is relatively stiff and relatively long (e.g., 55 d to 65 d over a few inches). However, in neurovascular catheters, an extremely soft polymer is typically used to bring the hardness down to about 25 d to 35 d over a similar length. In addition, the wall thickness of the catheter is very thin so that only 0.001 to 0.002-inch of polymer is actually in the wall that will allow a memory shape to be set.

The existing methods of curving catheters suffer from a number of disadvantages. First, the curved shape requires some rigidity of the catheter, particularly the polymer material, to maintain the curvature. Second, the soft wall of the catheter needed to make the curve shape often becomes crushed or kinked during use. Third, a small size and tight curvature of the catheter is difficult to achieve.

Thus, there is a need in the industry for an improved catheter having a curved distal end and method of making the same that will allow a small diameter and tight curvature to be achieved while still using a soft polymer material.

### SUMMARY OF THE INVENTION

It is an object of the present invention to provide a catheter having a curved distal end and method of making the same that overcomes the problems in the above-mentioned prior art.

It is a further object of the present invention to provide an improved catheter having a curved distal end and method of making the same that achieves a small diameter and tight curvature using a soft polymer material.

It is a further object of the present invention to provide an improved catheter having a curved distal end and method of making the same that is resistant to crushing and kinking at the curved distal portion.

To achieve the foregoing and other objects and in accordance with the purpose of the present invention, as embodied and broadly described herein, the present invention provides a catheter having a curved distal end in which a resilient fiber embedded in a polymer material of the catheter sidewall imparts a desired curvature in the distal end portion of the catheter. The resilient fiber has a helical coil shape with a series of helical coils disposed about a center line. During manufacturing, the resilient fiber is bent into a curved condition in which the center line is curved, and then the resilient fiber is heated while in its curved condition to create a memory set in the helical coil shape. The resilient fiber is then placed over a mandrel along with a fibrous reinforcement material, and a polymer material is applied over the mandrel to form a catheter with the resilient fiber embedded in the polymer material of the catheter sidewall. Upon removing the mandrel from the lumen of the catheter, the catheter will bend into a curved shape corresponding to the memory set in the resilient fiber.

According to a broad aspect of the present invention, a catheter is provided comprising: a tubular member having a sidewall formed of polymer material; and a resilient fiber embedded within the polymer material of the sidewall, the resilient fiber having a helical coil shape with a preset curvature that imparts a bend in the tubular member in its at-rest condition.

According to another broad aspect of the present invention, a catheter is provided, comprising: a tubular member having a lumen and a sidewall formed of a soft pliable material; and a resilient fiber embedded within the soft pliable material of the sidewall and extending circumferentially a plurality of turns around the lumen, the resilient fiber having a memory set therein that imparts a bend in the tubular member in its at-rest condition.

According to another broad aspect of the present invention, a method of manufacturing a catheter is provided, comprising the steps of: providing a resilient fiber having a helical coil shape comprising a series of helical coils disposed about a center line; bending a portion of the helical coil shape into a curved condition in which the center line thereof is curved; heating the resilient fiber to create a memory set in the helical coil shape in its curved condition; placing the resilient fiber over a mandrel; and applying a polymer material over the mandrel to form a tubular member having the resilient fiber embedded within a sidewall thereof.

Additional objects, advantages, and novel features of the invention will be set forth in the following description, and will become apparent to those skilled in the art upon reading this description or practicing the invention. The objects and advantages of the invention may be realized and attained by the appended claims.

### BRIEF DESCRIPTION OF THE DRAWINGS

The present invention will become more clearly appreciated as the disclosure of the present invention is made with reference to the accompanying drawings. In the drawings:

FIG. 1 shows a core mandrel over which a catheter will be constructed according to the present invention.

FIG. 2 shows a liner placed over the mandrel in the catheter manufacturing process of the present invention.

FIG. 3 shows a winding operation for applying a fibrous reinforcement over the mandrel and liner in the catheter manufacturing process of the present invention.

FIG. 4 shows the catheter liner and mandrel following the application of the fibrous reinforcement shown in FIG. 3.

FIG. 5 shows a resilient fiber wound into a helical coil having a generally straight center line.

FIG. 6 shows the resilient fiber of FIG. 5 with the center line of the helical coil having a bent configuration.

FIG. 7 shows the resilient fiber in its bent configuration being slid over the distal portion of the reinforced catheter liner shown in FIG. 4.

FIG. 8 shows the reinforced catheter liner with the resilient fiber slid over its distal portion.

FIG. 9 shows particulate materials being applied over the catheter liner, fibrous reinforcement, and resilient fiber according to the catheter manufacturing process of the present invention.

FIG. 10 shows the manufactured catheter of the present invention before the mandrel is removed from the lumen of the catheter.

FIG. 11 shows the curved shape of the catheter of the present invention after the mandrel is removed from the lumen of the catheter.

### DETAILED DESCRIPTION OF THE INVENTION

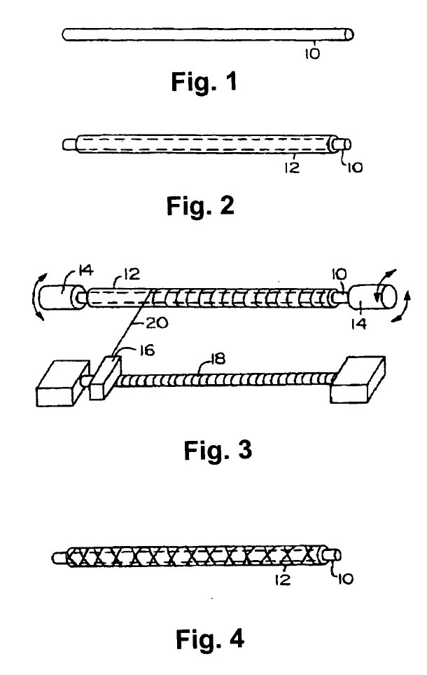

A catheter C having a curved distal end portion and a method of making the same according to the present invention will be described in detail hereinafter with reference to FIGS. 1 to 11 of the accompanying drawings.

The method of making a catheter C starts with a core mandrel 10, as shown in FIG. 1. The catheter C will be constructed over the core mandrel 10 using much of the same technology disclosed in the Applicant's prior U.S. Pat. No. 6,030,371, which is incorporated herein by reference.

A catheter liner 12 is placed over the core mandrel 10, as shown in FIG. 2. The liner 12 can be formed of a variety of different materials but is generally less than 20% of the intended wall thickness. As an example, a liner having a 0.00150 inch wall thickness of TFE can be used. Alternatively, the process of the present invention can be performed without a liner, whereby a polymer material is applied directly on the mandrel 10.

A reinforcement filament 20 is then applied over the liner 12, as shown in FIG. 3. During this operation, the mandrel/liner combination is loaded into rotating chucks 14. A filament winding head 16 travels on a screw carrier 18 longitudinally along the mandrel 10 to apply fibrous reinforcement filament 20 over the mandrel 10 at a winding angle range of 0 to 90 degrees relative to the longitudinal axis of the catheter C. For portions of the catheter C that require great circumferential rigidity or kink resistance, a very tight winding angle (e.g., 80 to 90 degrees) of the reinforcement filament 20 can be used, and for portions of the catheter C that require low rigidity, the reinforcement filament 20 can be applied in a low winding angle (e.g., 0 to 10 degrees). The winding angle of the reinforcement filament 20 can be continuously varied over the length of the catheter C by controlling the rotation speed of the mandrel 10 and the movement of the filament winding head 16 along the support 18. The catheter liner 12 and mandrel 10 having the reinforcement filament 20 applied thereover is shown in FIG. 4.

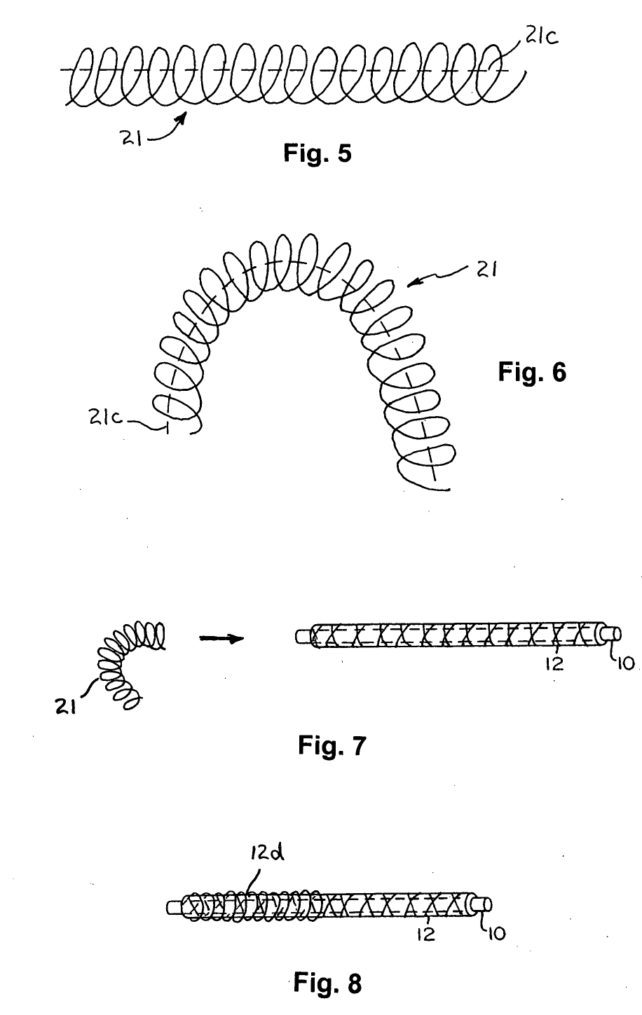

The catheter C is provided with a preset curvature using a resilient fiber 21 embedded in the polymer wall of the catheter C. For example, the resilient fiber 21 can be formed of a metallic steel heat-tempered spring alloy, such as a titanium-nickel-chromium alloy, or a boron fiber having a diameter of 0.001 to 0.006 inches. The resilient fiber 21 is separately prepared by winding the fiber 21 into a helical coil shape having a series of helical coils disposed about a center line 21c. As shown in FIG. 5, the center line 21c of the resilient fiber 21 is initially straight.

The resilient fiber 21 is then bent so that the center line 21c of the helical coil has a curved configuration, as shown in FIG. 6. The coiled resilient fiber 21 with the curved configuration is then heated to a temperature sufficient to create a heat set in the resilient fiber 21 so that the fiber remains in its curved configuration in its at-rest condition. That is, the coiled resilient fiber 21 is heated and then cooled to establish a memory set in the fiber 21 corresponding to the desired curved shape of the catheter C.

The coiled resilient fiber 21 is then slid over the distal portion of the reinforced catheter liner 12, as shown in FIGS. 7 and 8. Although the coiled resilient fiber 21 has a bent configuration (FIG. 7) before it is slid over the reinforced catheter liner 12, the mandrel 10 used during the manufacturing process is sufficiently rigid that the coiled resilient fiber 21 adopts a straight configuration (FIG. 8) once it is slid into position on the catheter liner 12. In an alternative embodiment, an additional layer of fibrous reinforcement material can be applied over the coiled resilient fiber 21 after it is slid into the position shown in FIG. 8.

After the reinforcement filament 20 and coiled resilient fiber 21 are applied over the catheter liner 12, the catheter C can be formed, for example, using the nonextrusion manufacturing method and apparatus described in the Applicant's U.S. Pat. No. 6,030,371. Using this method, the catheter C can be formed with a variable hardness and other properties over its length by continuously changing the constituents or mixtures of the polymer material(s) being used. The catheter C can thus have a relatively stiff or hard portion near its proximal end and a relatively softer portion near its distal end.

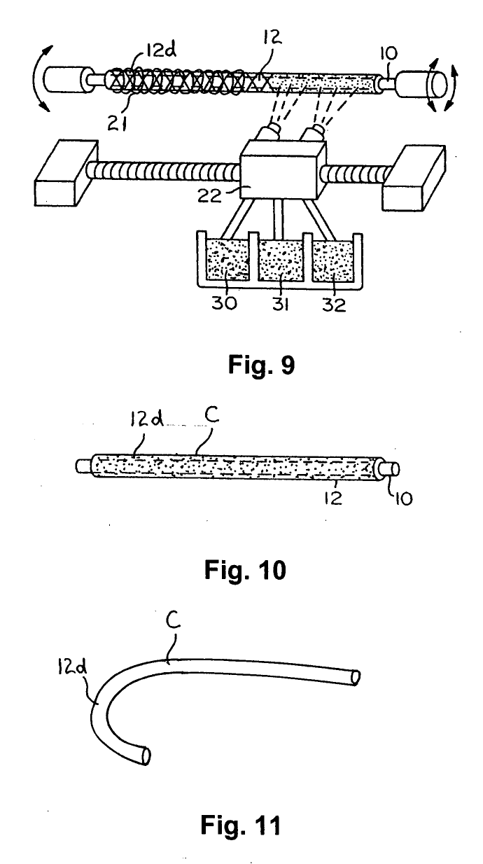

As shown in FIG. 9, an atomizing spray head 22 traverses the mandrel/liner and applies atomized sprays that fuse to the substrate surface the sprays impinge upon (i.e., the mandrel 10, the liner 12, the reinforcement fiber 20, the coiled resilient fiber 21, or the previous layer of polymer material). The substrate can be preheated to ensure complete fusion of the sprayed polymer to the substrate. This preheating can be accomplished with infrared, hot air, or resistance heating of the core mandrel 10 or other suitable means.

A suitable atomizing spray head 22 according to the present invention is described in detail in the Applicant's prior U.S. Pat. No. 6,030,371. The atomizing spray head 22 is connected to multiple containers 30 and 31 of polymer materials having varying degrees of hardness or other desired properties. The atomizing spray head 22 can also be connected to a container 32 of an opacifier material, such as tungsten.

While the mandrel/liner is spinning, the atomizing spray head 22 traverses a path parallel to the axis of the rotating mandrel/liner. As it traverses this path, a metering valve (not shown) can be set such that only the harder polymer (e.g., from the container 30) is applied at what will be the proximal end of the catheter C. As the head 22 traverses the mandrel/liner, the metering valve is controlled such that it ports to the harder polymer to a lesser degree and to the softer polymer (e.g., from the container 31) to a higher degree until finally only the softest polymer is applied at the distal end portion 12d of the catheter C, which will serve as the curved distal end portion of the catheter C. The different hardness polymer materials used in the present invention can be colored to provide visual confirmation of the transition of hardness.

In a similar fashion, opacifying powder can be selectively applied from the container 32. In one example, a single layer of polymer material can be applied over the fibrous reinforcement 20 and the coiled resilient fiber 21. The single layer of polymer material can be followed by a layer of opacifier material and another layer of polymer material. The movement of the head 22 can be paused momentarily to apply circumferential rings of high opacifier concentration, which serve as markers when the catheter C is used under X-ray.

Once the particulate material has been applied to coat the entire outer surface of the catheter C, the coated liner 12 and mandrel 10 are then heated (e.g., baked in an oven) to consolidate the particulate material. The manufactured catheter C with the straight mandrel 10 still in the lumen thereof is shown in FIG. 10.

After the particulate material is consolidated, the mandrel 10 is removed from the lumen of the catheter C. With the mandrel 10 removed, the distal end portion 12d of the catheter C adopts a curved shape, as shown in FIG. 11, due to the preset curvature in the coiled resilient fiber 21 embedded in the polymer material of the catheter wall.

The coiled resilient fiber 21 embedded in the polymer wall allows the catheter C to be manufactured without imparting a preset curvature or “memory” into the polymer material of the catheter C. The preset curvature is contained in the coiled resilient fiber 21 instead of the polymer material. As a result, an extremely soft polymer material can be used at the distal end portion 12d of the catheter C. For example, a nylon, urethane, PE, TFE, or other suitable polymer material can be used, which is very soft and offers little resistance to the preformed shape of the resilient fiber 21. Moreover, the helical coils of the resilient fiber 21 resist kinking and crushing, thereby allowing very small diameters and tight bending radii to be attained at the distal end portion 12d of the catheter C.

In use, the catheter C according to the present invention can be straightened over a guide wire to enter a patient's body. Once in the body, the catheter C is advanced over the guide wire until it reaches a soft portion of the guide wire near the diseased section of the patient's body. At this point, the memory set in the embedded resilient fiber 21 overrides the straightening force of the guide wire and starts to bend into its preset curved shape, as shown in FIG. 11. The catheter C with its curved distal end can be optimally positioned for transmitting diagnostic and therapeutic devices or media into the vascular system.

The preset curved shape of the embedded resilient fiber 21 is not limited to a 180-degree bend or a single bend, as shown in FIGS. 6 and 11. Instead, multiple bends or bends of different angles can be used to deflect the catheter C in a specific way for a given application or procedure. Moreover, the catheter C of the present invention can be made using other known manufacturing techniques, such as extrusion of a polymer material over the fibrous reinforcement 20 and the coiled resilient fiber 21.

While the invention has been specifically described in connection with specific embodiments thereof, it is to be understood that this is by way of illustration and not of limitation, and the scope of the appended claims should be construed as broadly as the prior art will permit.

What is claimed is:

1. A catheter comprising:
a tubular member having a sidewall formed of polymer material; and
a resilient fiber embedded within the polymer material of said sidewall, said resilient fiber having a helical coil shape with a preset curvature that imparts a bend in said tubular member in its at-rest condition.

2. The catheter according to claim 1, wherein said resilient fiber is embedded within the polymer material only in a distal end portion of the tubular member.

3. The catheter according to claim 1, further comprising a fibrous reinforcement material embedded within the polymer material.

4. The catheter according to claim 1, wherein said helical coil shape of said resilient fiber comprises a series of helical coils disposed about a center line, and said preset curvature of said resilient fiber comprises a portion of said helical coil shape in which the center line is curved.

5. The catheter according to claim 1, wherein said polymer material is extremely soft and does not have a significant memory set therein that would impart a bend in the tubular member in its at-rest condition.

6. The catheter according to claim 1, wherein said resilient fiber comprises a metallic steel heat-tempered spring alloy.

7. The catheter according to claim 1, wherein said resilient fiber comprises a boron fiber.

8. The catheter according to claim 1, wherein said resilient fiber has a diameter of 0.001 to 0.006 inches.

9. A catheter comprising:
a tubular member having a lumen and a sidewall formed of a soft pliable material; and
a resilient fiber embedded within the soft pliable material of said sidewall and extending circumferentially a plurality of turns around said lumen, said resilient fiber having a memory set therein that imparts a bend in said tubular member in its at-rest condition.

10. The catheter according to claim 9, wherein said resilient fiber has a helical coil shape with a preset curvature that curves about a longitudinal center line of said helical coil shape.

11. The catheter according to claim 9, wherein said soft pliable material is a polymer material.

12. The catheter according to claim 9, further comprising a fibrous reinforcement material embedded within the soft pliable material.

13. The catheter according to claim 9, wherein said resilient fiber has a helical coil shape comprising a series of helical coils disposed about a center line, and said memory set in said resilient fiber comprises a portion of said helical coil shape in which the center line is curved.

14. The catheter according to claim 9, wherein said soft pliable material does not have a significant memory set therein that would impart a bend in the tubular member in its at-rest condition.

15. The catheter according to claim 9, wherein said resilient fiber comprises a metallic steel heat-tempered spring alloy.

16. The catheter according to claim 9, wherein said resilient fiber comprises a boron fiber.

17. A method of manufacturing a catheter, comprising the steps of:
providing a resilient fiber having a helical coil shape comprising a series of helical coils disposed about a center line;
bending a portion of said helical coil shape into a curved condition in which the center line thereof is curved;
heating said resilient fiber to create a memory set in said helical coil shape in its curved condition;
placing said resilient fiber over a mandrel; and
applying a polymer material over said mandrel to form a tubular member having said resilient fiber embedded within a sidewall thereof.

18. The method according to claim 17, further comprising the step of removing the mandrel from said tubular member and allowing the tubular member to bend into a curved shape corresponding to the memory set in said resilient fiber.

19. The method according to claim 17, further comprising the step of winding or braiding a fibrous reinforcement material over the mandrel before said resilient fiber is placed over the mandrel.

20. The method according to claim 17, wherein said resilient fiber is placed over the mandrel in a location corresponding to a distal end portion of the catheter.
